(19) United States
(12) Patent Application Publication
(10) Pub. No.: US 2022/0299221 A1
(43) Pub. Date: Sep. 22, 2022

(54) PLASMA DISINFECTION DEVICE

(71) Applicant: KABUSHIKI KAISHA TOSHIBA, Tokyo (JP)

(72) Inventors: Akio UI, Suginami (JP); Yosuke SATO, Kawasaki (JP); Shotaro OKA, Shinjuku (JP)

(73) Assignee: KABUSHIKI KAISHA TOSHIBA, Tokyo (JP)

(21) Appl. No.: 17/465,139

(22) Filed: Sep. 2, 2021

(30) Foreign Application Priority Data
Mar. 22, 2021 (JP) 2021-047886

(51) Int. Cl. F24F 8/20; F24F 8/95; F24F 13/30

(52) U.S. Cl. CPC F24F 8/20; F24F 8/95; F24F 13/30

(57) ABSTRACT

A plasma disinfection device of an embodiment includes: an electrical dust collector including a plurality of heat exchange fins, a needle electrode which causes a discharge in a gas flow flowing between the plurality of heat exchange fins, and a direct-current power supply electrically connected to the needle electrode; and a plasma generator including a dielectric provided on each of facing surfaces of the plurality of heat exchange fins, a discharge electrode provided to be exposed on a surface of the dielectric and arranged to cross a direction of flow of the gas flow, and an alternating-current power supply electrically connected to the discharge electrode.

### CROSS REFERENCE TO RELATED APPLICATION

This application is based upon and claims the benefit of priority from Japanese Patent Application No. 2021-047886, filed on Mar. 22, 2021; the entire contents of which are incorporated herein by reference.

### FIELD

Embodiments disclosed herein relate to a plasma disinfection device.

### BACKGROUND

Fine particles such as PM2.5 (fine particulate matter with a particle size of 2.5 μm or less) and PM0.1 (fine particulate matter with a particle size of 0.1 μm in size) which are floating in the atmosphere are likely to enter pulmonary alveoli and be deposited thereon, which causes a concern about adverse effects on a human body. Environmental standards are set for these, and a concentration distribution once every hour is announced from Ministry of the Environment, Japan, as one of monitored substances. Further, recently, resulting from air pollution problems in China, a concern also arises over flying of PM2.5 from China to Japan. As health concerns expand, it becomes more important to clean air at home, in offices, and the like. An air-conditioner having a function of collecting and removing the fine particles in the atmosphere actively by a filter method or an electrical dust collection method is also produced. For example, as an electrical dust collector mounted on the air-conditioner, there is known a method in which the fine particles in the atmosphere are collected on a heat exchange fin to be washed away with condensed drain water, to which attention is paid as a high-efficiency dust collector whose pressure loss is small and which is maintenance-free.

Meanwhile, as infection control measures, it also becomes important to remove droplets from a mouse of an infected person droplets, and, viruses (being each 0.1 μm in size) and bacteria (being each 1 μm in size) which are floating in the atmosphere. Here, the removals of viruses and bacteria are mentioned as disinfection. Both a virus and a bacterium are a kind of fine particles, and are verified to be able to be collected by the electrical dust collector. Moreover, most viruses in the atmosphere are also said to adhere to fine particles such as PM2.5 and droplets from a mouse. That is, by the above-described electrical dust collector, viruses are collected on the heat exchange fin directly or as fine-particle adhering matter.

An electrical dust collection-type air-conditioner which collects fine particles such as PM2.5 by using the heat exchange fin collects floating viruses and bacteria in air directly as fine particles or together with fine particles, thereby removing them from the atmosphere. However, there is a concern that the collected viruses and bacteria re-float from the heat exchange fin or flow out to the outside in a state of being contained in the drain water. Further, because the floating viruses and the floating bacteria which are not collected despite adhering to the fine particles or pass through between the heat exchange fins without adhering thereto are also capable of existing, the efficiency of disinfecting them is required to be enhanced.

### BRIEF DESCRIPTION OF THE DRAWINGS

FIG. 1 is a perspective view illustrating a plasma disinfection device of a first embodiment.

FIG. 2 is a sectional view of the plasma disinfection device illustrated in FIG. 1.

FIG. 3 is a sectional view enlarging and illustrating a part of the plasma disinfection device illustrated in FIG. 1.

FIG. 4 is a sectional view illustrating a plasma disinfection device of a second embodiment.

FIG. 5 is a sectional view illustrating a plasma disinfection device of a third embodiment.

FIG. 6 is a sectional view illustrating a plasma disinfection device of a fourth embodiment.

FIG. 7 is a sectional view enlarging and illustrating a part of the plasma disinfection device illustrated in FIG. 6.

FIG. 8 is a sectional view illustrating a plasma disinfection device of a fifth embodiment.

FIG. 9 is a sectional view enlarging and illustrating a part of the plasma disinfection device illustrated in FIG. 8.

FIG. 10 is a sectional view illustrating a plasma disinfection device of a sixth embodiment.

### DETAILED DESCRIPTION

A plasma disinfection device of an embodiment includes: an electrical dust collector including a plurality of heat exchange fins, a needle electrode which causes a discharge in a gas flow flowing between the plurality of heat exchange fins, and a direct-current power supply electrically connected to the needle electrode; and a plasma generator including a dielectric provided on each of facing surfaces of the plurality of heat exchange fins, a discharge electrode provided to be exposed on a surface of the dielectric and arranged to cross a direction of flow of the gas flow, and an alternating-current power supply electrically connected to the discharge electrode.

Hereinafter, plasma disinfection devices of embodiments will be described with reference to the drawings. Note that in each embodiment, substantially the same components are denoted by the same reference signs, and descriptions thereof are sometimes partly omitted. The drawings are schematic, and a relation between a thickness and a planar dimension of each component, a ratio of thicknesses of the respective components, and the like are sometimes different from actual ones.

### First Embodiment

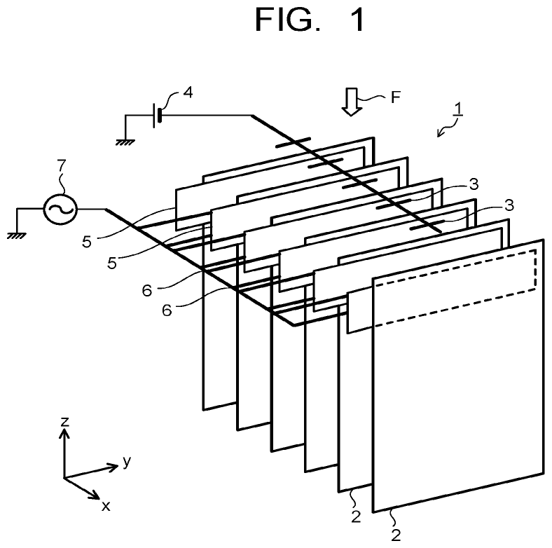

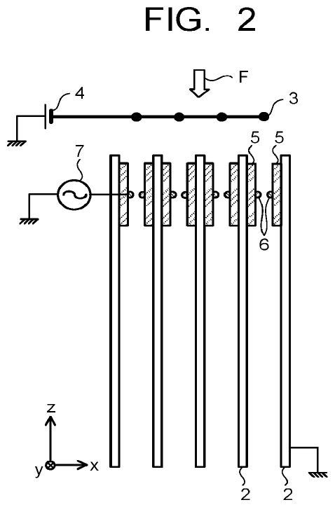

FIG. 1 is a perspective view illustrating a plasma disinfection device of a first embodiment, and FIG. 2 is a sectional view illustrating the plasma disinfection device of the first embodiment. A plasma disinfection device 1 illustrated in FIG. 1 and FIG. 2 includes an electrical dust collector including a plurality of heat exchange fins 2, a needle electrode 3, and a direct-current power supply 4, and a plasma generator including a plurality of dielectrics 5 each provided on each of facing surfaces of the plurality of heat exchange fins 2, a discharge electrode 6 provided to be exposed on a surface of each of the plurality of dielectrics 5, and an alternating-current power supply 7. In FIG. 1 and FIG. 2, a parallel arrangement direction of the plurality of heat exchange fins 2 is set as an x direction, a depth direction of the heat exchange fin 2 (a direction orthogonal to the x direction) is set as a y direction, and a height direction of the heat exchange fin 2 orthogonal to the x direction and the y direction (a flow direction of gas flow) is set as a z direction.

The electrical dust collector is one installed in an air conditioning system such as, for example, an air-conditioner including a heat exchanger, and the plurality of heat exchange fins 2 constitute a part of the heat exchanger. Although illustration is omitted in FIG. 1 and FIG. 2, a passage of a heat medium such as a refrigerant or a heating medium (medium passage) is provided in the heat exchange fins 2 or outside them, and the heat medium circulating in the medium passage circulates via not-illustrated compressor, condenser, evaporator, and the like. The heat medium flowing in the plurality of heat exchange fins 2 or outside them performs heat exchange between the heat medium and a gas flow F (indicated by an arrow F in each of FIG. 1 and FIG. 2) such as airflow flowing in the z direction between the plurality of heat exchange fins 2, which causes the gas flow F to be cooled or heated, thereby adjusting the gas flow F to a desired temperature.

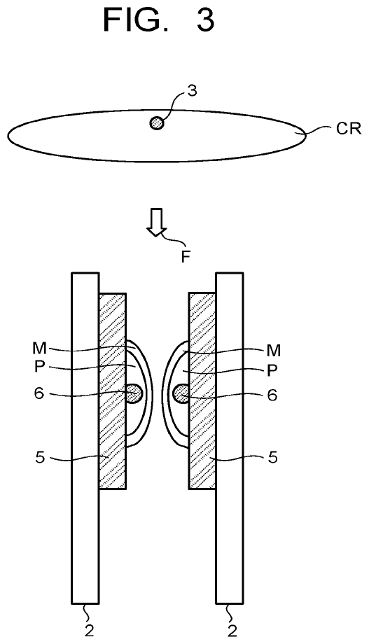

The electrical dust collector has the needle electrode 3 arranged on an upstream side further than the heat exchange fins 2 of the gas flow F flowing between the plurality of heat exchange fins 2 arranged in parallel. The needle electrode 3 is electrically connected to the direct-current power supply 4. The heat exchange fin 2 is grounded. As illustrated in FIG. 3, applying a high-voltage negative voltage from the direct-current power supply 4 to the needle electrode 3 causes a corona discharge at the tip of the needle electrode 3, and electrons, ions, and so on generated there form a charged region CR above the plurality of heat exchange fins 2 while spreading around the needle electrode 3. The electrons, the ions, and so on formed in the charged region CR adhere to PM2.5 and the other floating fine particles, droplets, viruses, bacteria, and further, fine particles such as floating fine particles and droplets to which the viruses and the like adhere, which are contained in the gas flow F passing through the charged region CR, resulting in that they are negatively charged. The charged fine particles ride the gas flow and flow in a direction of the heat exchange fin 2, and at the same time, receive power from the negative voltage applied to the needle electrode 3 to move in the x direction, and collide with the heat exchange fin 2, resulting in being collected from an upstream side on the heat exchange fin 2. The charged region CR formed by the corona discharge or the like caused by the needle electrode 3 allows the fine particles and the like to be charged, but fails to sterilize, for example, the viruses, the bacteria and the like adhering to the fine particles due to low energy. The electrical dust collector only exhibits a dust collection function.

The plasma generator is one which functions as a dielectric barrier discharge (DBD) device, and has the plurality of dielectrics (layers) 5 each provided on each of facing surfaces of the plurality of heat exchange fins 2. The discharge electrode (first electrode) 6 is provided to be exposed on a surface of each of the plurality of dielectrics 5. The discharge electrode 6 is electrically connected to the alternating-current power supply 7. The heat exchange fin 2 is generally constituted by a conductive material such as a metal material having corrosion resistance. Accordingly, the heat exchange fin 2, which is grounded (0V), arranged to face the discharge electrode (first electrode) 6 with the dielectric 5 interposed therebetween is one which functions as a second electrode of the DBD device.

The discharge electrode 6 is provided on the surface of the dielectric 5, and has a shape such as a wire shape, a bar shape, a plate shape, or a foil shape. The discharge electrode 6 is exposed on the dielectric 5, and extends in the y direction. That is, the discharge electrode 6 extends in a direction crossing the flow direction of the gas flow F (arrow F direction), for example, a direction orthogonal thereto. As illustrated in FIG. 3, a plasma P formed around the discharge electrode 6 disinfects and further sterilizes viruses, bacteria, and the like which adhere on the heat exchange fin 2 together with fine particles or independently thereof. For this reason, the discharge electrode 6 is preferably arranged on the upstream side of the gas flow F flowing between the plurality of heat exchange fins 2, in other words, on an end portion side of each of the plurality of heat exchange fins 2 located on the upstream side of the gas flow F. The discharge electrode 6 is preferably arranged in a range of 5 mm or more and 15 mm or less from the upstream-side end portion of the heat exchange fin 2. Because the fine particles collected on the heat exchange fin 2 are likely to be collected in such a region of the heat exchange fin 2 as described above, it is possible to enhance an effect of the plasma P formed around the discharge electrode 6.

For the dielectric 5, for example, there is used a glass material such as alkali-free glass or borosilicate glass, a ceramic material such as alumina ceramics or silicon nitride ceramics, a resin material such as an epoxy resin or a polyether resin, or the like. For the discharge electrode (first electrode) 6, for example, there is used a metal material such as copper, silver, chromium, titanium, or platinum. Further, for the heat exchange fin 2 functioning as the second electrode, the metal material similar to the constituent material of the discharge electrode 6 may be used, or an alloy material or the like having corrosion resistance and conductivity may be used. The heat exchange fin 2 is preferably constituted by the metal material or the like having the conductivity and the corrosion resistance against contact with the gas flow F. As a waveform of a voltage applied to the discharge electrode 6, an alternating-current waveform or a pulse waveform is used. A frequency of the alternating current can be used from several Hz to several GHz. The frequency of the alternating current is typically from several kHz to several MHz, and a microwave of GHz order can also be used. A commercial power supply frequency (50 or 60 Hz) is also usable. As the pulse waveform, a waveform having a rise time from several nanoseconds to several hundreds of microseconds can be used.

Applying voltage from the alternating-current power supply 7 to the discharge electrode 6 provided in such a state as to be exposed on the dielectric 5 causes a dielectric breakdown between the discharge electrode (first electrode) 6 and the heat exchange fin 2 functioning as the second electrode which are arranged with the dielectric 5 interposed therebetween, which generates the plasma P. The surface plasma P is formed around the discharge electrode (first electrode) 6 along the surface of the dielectric 5. In consideration of formability of the plasma P, an interval in the x direction between the plurality of heat exchange fins 2 is preferably set to 1.5 mm or more and 8 mm or less, and further preferably set to 2 mm or more and 5 mm or less. Providing the dielectric 5 and discharge electrode 6 on each of the facing surfaces of the plurality of heat exchange fins 2 arranged at such intervals makes the plasma P likely to be generated, and makes plasma induced flow likely to be produced. A thickness of the dielectric 5 is preferably thinned to make a discharge likely to be generated, but excessively thinning easily causes a reduction in durability, or the like. In consideration of such a point as described above, the thickness of the dielectric 5 is preferably set to 0.3 mm or more and 1.5 mm or less.

As described above, applying voltage from the alternating-current power supply 7 to the discharge electrode 6 causes the plasma (surface plasma) P to be formed around the discharge electrode 6 along the surface of the dielectric 5 by a DBD. The fine particles or the like (indicated by M in FIG. 3) collected by the electrical dust collector are pulled toward the plasma P, and pass through the inside thereof. Because active oxygen (excited-state oxygen atoms, excited-state oxygen molecules, and the like), OH radicals, ozone, electrons, ultraviolet rays, and so on are generated in the plasma P, these can disinfect (inactivate) and further sterilize (destroy) the viruses, the bacteria, and the like adhering to the fine particles and the like collected on the heat exchange fin 2 by the needle electrode 3. In general, because viruses and bacteria in the atmosphere disperse at a small presence density, only generating the plasma results in that a space volume of the plasma is also small, thus making it difficult to enhance disinfection efficiency of viruses and bacteria. In contrast with this, by being collected as an individual to be thereafter disinfected and further sterilized by the plasma P, the disinfection efficiency can be dramatically enhanced.

Moreover, owing to the DBD formed by the discharge electrode (first electrode) 6, the dielectric 5, and the heat exchange fin 2 functioning as the second electrode, induced flow is produced to pull the atmosphere into the plasma P. For this reason, the atmosphere flowing between the heat exchange fins 2 is pulled into the plasma P, and viruses and bacteria in the atmosphere can be disinfected directly or with a form of adhering to fine particles remaining. A discharge between the heat exchange fins 2 arranged with a narrow gap allows the plasma P to be efficiently generated, and the atmosphere flowing between the heat exchange fins 2 to be efficiently passed in the plasma P. Consequently, the viruses and the bacteria in the atmosphere can be efficiently disinfected. Note that a disinfection process is also allowed to be set aside from a dust collecting process as described later, and the operation can be performed under advantageous conditions depending on the respective set conditions.

### Second Embodiment

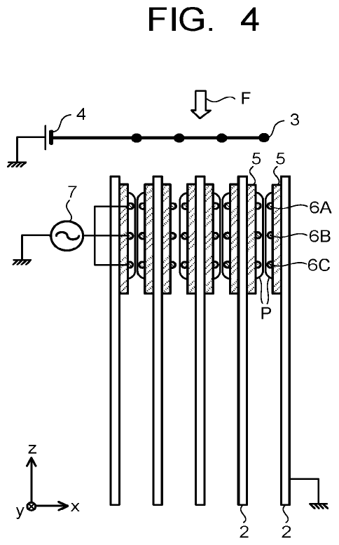

Next, a plasma disinfection device of a second embodiment will be described with reference to FIG. 4. In a plasma disinfection device 1 illustrated in FIG. 4, on a surface of each of a plurality of dielectrics 5 provided on facing surfaces of a plurality of heat exchange fins 2, a plurality of discharge electrodes 6 (6A, 6B, 6C) are each arranged in parallel along a flow direction of a gas flow F (F direction) so as to extend in a direction crossing the flow direction of the gas flow F (arrow F direction). The plasma disinfection device of the second embodiment has a configuration similar to that of the plasma disinfection device of the first embodiment except that the plurality of discharge electrodes 6A, 6B, 6C are arranged on the surface of the dielectric 5. Arranging the plurality of discharge electrodes 6A, 6B, 6C on the dielectric 5 makes it possible to extend a formation region of a plasma P. Consequently, the gas flow F such as the atmosphere flowing between the heat exchange fins 2 can be passed efficiently in the plasma P. This allows viruses and bacteria in the atmosphere to be more efficiently disinfected.

### Third Embodiment

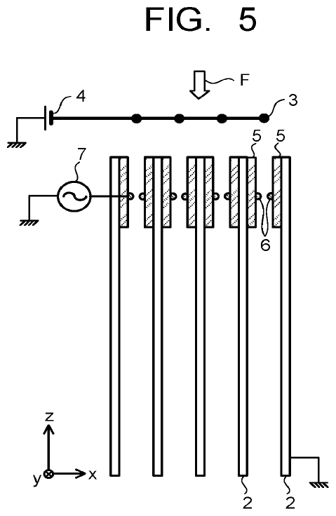

Next, a plasma disinfection device of a third embodiment will be described with reference to FIG. 5. In a plasma disinfection device 1 illustrated in FIG. 5, a plurality of dielectrics 5 each provided on each of facing surfaces of a plurality of heat exchange fins 2 are extended to end portions of the plurality of heat exchange fins 2 located on an upstream side of a gas flow F. Thus, by covering the end portion located on the upstream side of the gas flow F of each of the plurality of heat exchange fins 2 with each of the plurality of dielectrics 5, it is possible to suppress an abnormal discharge generated between a discharge electrode 6 and the exposed heat exchange fin 2. Also in a case where the gas flow F contains conductive fine particles, the abnormal discharge can be suppressed. That is, the plasma can be formed more stably along a surface of the dielectric 5 around the discharge electrode 6. Consequently, it becomes possible to disinfect viruses and bacteria in the atmosphere more stably and repeatably. The plasma disinfection device of the third embodiment has a configuration similar to that of the plasma disinfection device of the first embodiment except that the dielectric 5 is extended to the end portion of the heat exchange fin 2.

### Fourth Embodiment

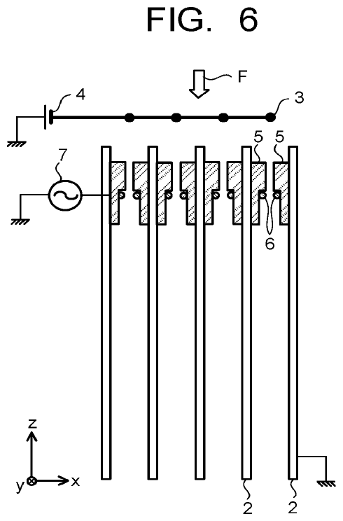

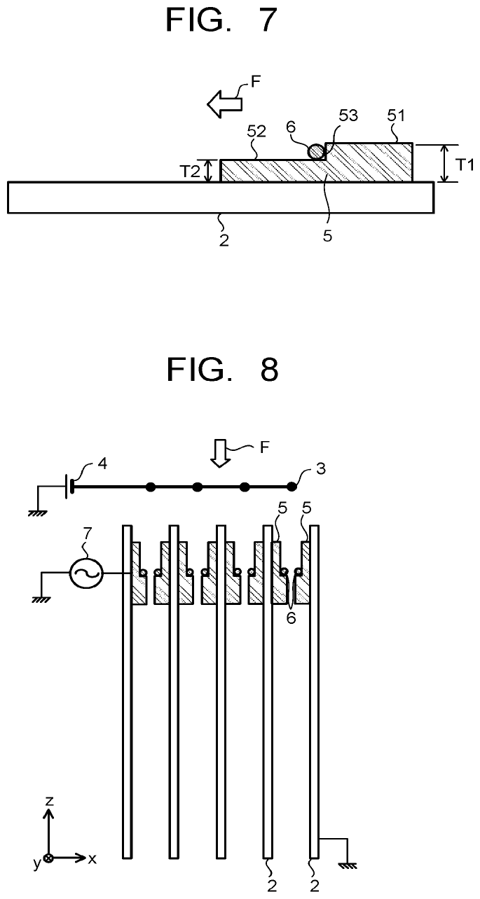

Next, a plasma disinfection device of a fourth embodiment will be described with reference to FIG. 6. In a plasma disinfection device 1 illustrated in FIG. 6, in a shape of a plurality of dielectrics 5 each provided on each of facing surfaces of a plurality of heat exchange fins 2, a portion located on an upstream side of a gas flow F is deformed, as enlarged and illustrated in FIG. 7. The dielectric 5 has a stepped shape so that the portion located on the upstream side of the gas flow F of a discharge electrode 6 is covered with the dielectric 5. That is, the dielectric 5 has a shape in which a first step portion 51 provided on the upstream side of the gas flow F and having a thickness T1 and a second step portion 52 provided on a downstream side of the gas flow F and having a thickness T2 smaller than the thickness T1 (T2<T1) are connected by a stepped surface 53. The discharge electrode 6 is arranged along the stepped surface 53. Since the stepped surface 53 of the dielectric 5 is present on the upstream side of the discharge electrode 6, the upstream side of the discharge electrode 6 is covered with the dielectric 5. In this occasion, not the stepped shape, but a shape in which the upstream side of the discharge electrode 6 is covered with the dielectric 5, for example, a streamline shape in which a coating-type dielectric is coated and fixed on the upstream side of the discharge electrode 6 is applicable.

Applying the dielectric 5 having such a shape makes a plasma ion flow based on plasma formed by the discharge electrode 6 likely to be produced from the upstream side toward the downstream side. Accordingly, a pressure loss of the gas flow F due to the plasma generator is suppressed, and this allows the gas flow F to flow easily and a gas flow amount increases between the plurality of heat exchange fins 2. That is, this contributes to mitigating the pressure loss due to the heat exchange fin 2, and, the dielectric 5 and the discharge electrode 6 installed between the heat exchange fins 2. In addition, this further contributes to improve efficiency of heat exchange of a heat exchange action of the gas flow F by using the plurality of heat exchange fins 2, efficiency of fine particle collection of a particle collection action by a corona discharge, efficiency of disinfection of a disinfection action of viruses and bacteria by a DBD. Note that except the shape of the above-described dielectric 5, the plasma disinfection device of the fourth embodiment has a configuration similar to that of the plasma disinfection device of the first embodiment.

### Fifth Embodiment

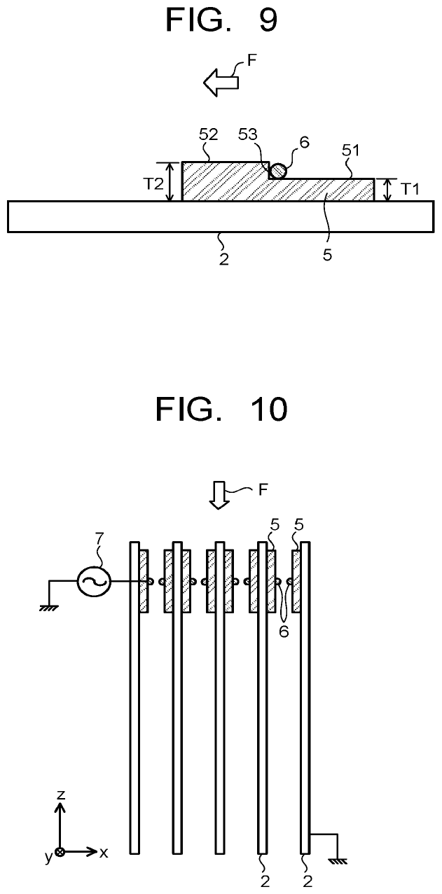

Next, a plasma disinfection device of a fifth embodiment will be described with reference to FIG. 8. In a plasma disinfection device 1 illustrated in FIG. 8, in a shape of a plurality of dielectrics 5 each provided on each of facing surfaces of a plurality of heat exchange fins 2, a portion located on a downstream side of a gas flow F is deformed, as enlarged and illustrated in FIG. 9. The dielectric 5 has a stepped shape so that the portion located on the downstream side of the gas flow F of a discharge electrode 6 is covered with the dielectric 5. The stepped shape of the dielectric 5 is deformed in the opposite direction to that in FIG. 7. That is, the dielectric 5 has a shape in which a first step portion 51 provided on an upstream side of the gas flow F and having a thickness T1 and a second step portion 52 provided on the downstream side of the gas flow F and having a thickness T2 larger than the thickness T1 (T2>T1) are connected by a stepped surface 53. The discharge electrode 6 is arranged along the stepped surface 53. Since the stepped surface 53 of the dielectric 5 is present on the downstream side of the discharge electrode 6, the downstream side of the discharge electrode 6 is covered with the dielectric 5.

Applying the dielectric 5 having such a shape makes a plasma ion flow based on plasma formed by the discharge electrode 6 likely to be produced from the downstream side toward the upstream side. Accordingly, fine particles charged by an electrical dust collector are likely to be collected on an upstream side of the discharge electrode 6. This allows suppression of outflow of the charged fine particles to the downstream side without being collected. However, because only disinfection processing with the plasma with a flow of the gas flow F maintained makes a gas flow amount decrease, and makes dust collection efficiency likely to decrease, it is preferable to stop flow of the gas flow F or to decrease the gas flow amount in a certain time cycle for a time carrying out an inactivation process with the plasma of fine particles collected on the upstream side of the discharge electrode 6 and a disinfection process of viruses and bacteria. Note that except such a shape of the plurality of dielectrics 5, the plasma disinfection device of the fifth embodiment has a configuration similar to that of the plasma disinfection device of the first embodiment.

### Sixth Embodiment

Next, a plasma disinfection device of a sixth embodiment will be described with reference to FIG. 10. In a plasma disinfection device 1 illustrated in FIG. 10, a needle electrode 3 and a direct-current power supply 4 constituting an electrical dust collector are not provided, but, except the above, has a configuration similar to that of the plasma disinfection device of the first embodiment. As previously described, it is efficient to disinfect viruses, bacteria, and the like collected by the electrical dust collector with plasma, but it sometimes becomes disadvantageous to install the electrical dust collector depending on a device configuration, a form of usage, or the like of a heat exchanger. In such a case, the disinfection of viruses, bacteria, and the like floating in the atmosphere may be performed by only a plasma generator including a plurality of dielectrics 5 each provided on each of facing surfaces of a plurality of heat exchange fins 2, a discharge electrode 6 provided to be exposed on a surface of each of the plurality of dielectrics 5, and an alternating-current power supply 7. Thus, it is possible to carry out the disinfection under advantageous conditions depending on set conditions, operating conditions, or the like of the heat exchanger.

While certain embodiments of the present invention have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

What is claimed is:

1. A plasma disinfection device comprising:
an electrical dust collector including a plurality of heat exchange fins, a needle electrode which causes a discharge in a gas flow flowing between the plurality of heat exchange fins, and a direct-current power supply electrically connected to the needle electrode; and
a plasma generator including a dielectric provided on each of facing surfaces of the plurality of heat exchange fins, a discharge electrode provided to be exposed on a surface of the dielectric and arranged to cross a direction of the gas flow, and an alternating-current power supply electrically connected to the discharge electrode.

2. The device according to claim 1, wherein the discharge electrode is arranged on an end portion side of each of the plurality of heat exchange fins located on an upstream side of the gas flow.

3. The device according to claim 2, wherein the discharge electrode is arranged in a range of 5 mm or more and 15 mm or less from the end portion of each of the plurality of heat exchange fins.

4. The device according to claim 2, wherein the dielectric extends to the end portion of each of the plurality of heat exchange fins.

5. The device according to claim 1, wherein a portion located on an upstream side of the gas flow of the discharge electrode is covered with the dielectric.

6. The device according to claim 5,
wherein the dielectric has a shape including a first step portion provided on an upstream side of the gas flow and having a thickness T1, a second step portion provided on a downstream side of the gas flow and having a thickness T2 smaller than the thickness T1, and a stepped surface connecting the first step portion and the second step portion, and
wherein the discharge electrode is arranged along the stepped surface of the dielectric.

7. The device according to claim 1, wherein a portion located on a downstream side of the gas flow of the discharge electrode is covered with the dielectric.

8. The device according to claim 7,
wherein the dielectric has a shape including a first step portion provided on an upstream side of the gas flow and having a thickness T1, a second step portion provided on a downstream side of the gas flow and having a thickness T2 larger than the thickness T1, and a stepped surface connecting the first step portion and the second step portion, and
wherein the discharge electrode is arranged along the stepped surface of the dielectric.

9. The device according to claim 1, wherein the plurality of heat exchange fins are arranged so that an interval between the facing surfaces is 1.5 mm or more and 8 mm or less.

10. The device according to claim 1, wherein the plasma generator includes, on a surface of the one dielectric, a plurality of the discharge electrodes arranged in parallel with the direction of the gas flow so as to cross the direction of the gas flow.

11. The device according to claim 1, wherein the plasma generator is configured to disinfect or sterilize viruses and bacteria adhering around fine particles collected on the plurality of heat exchange fins or viruses and bacteria captured independently on the plurality of heat exchange fins with a plasma formed around the discharge electrode.

12. A plasma disinfection device comprising:
a plurality of heat exchange fins;
a dielectric provided on each of facing surfaces of the plurality of heat exchange fins;
a discharge electrode provided to be exposed on a surface of the dielectric and arranged to cross a direction of the gas flow; and
an alternating-current power supply electrically connected to the discharge electrode.
